(19) United States
(12) Patent Application Publication
(10) Pub. No.: US 2024/0255598 A1
(43) Pub. Date: Aug. 1, 2024

(54) MRI APPARATUS, CONTROL METHOD FOR MRI APPARATUS, AND NON-TRANSITORY COMPUTER-READABLE STORAGE MEDIUM STORING CONTROL PROGRAM OF MRI APPARATUS

(71) Applicant: CANON MEDICAL SYSTEMS CORPORATION, Otawara-shi (JP)

(72) Inventors: Hiroyuki HATAKENAKA, Nasushiobara (JP); Daiki TAKEUCHI, Otawara (JP); Yugo TABATA, Nasushiobara (JP); Shinsuke KOMAKI, Otawara (JP)

(73) Assignee: CANON MEDICAL SYSTEMS CORPORATION, Otawara-shi (JP)

(21) Appl. No.: 18/423,393

(22) Filed: Jan. 26, 2024

(30) Foreign Application Priority Data
Jan. 31, 2023 (JP) 2023-013539

(51) Int. Cl. G01R 33/54; G01R 33/28; G01R 33/36

(52) U.S. Cl. CPC G01R 33/543; G01R 33/283; G01R 33/3664

(57) ABSTRACT

In one embodiment, An MRI apparatus comprising processing circuitry configured to: acquire an optical image of an object placed on a table of a bed; acquire information on an examination portion of the object; and select a specific RF coil to be used for imaging the examination portion of the object from a plurality of available RF coils based on the optical image and information on the plurality of available RF coils specified from the information on the examination portion.

### CROSS-REFERENCE TO RELATED APPLICATION

This application claims the benefit of priority of Japanese Patent Application No. 2023-13539, filed on Jan. 31, 2023, the entire contents of which are incorporated herein by reference.

### FIELD

Disclosed Embodiments relate to a magnetic resonance imaging (MRI) apparatus, a control method for an MRI apparatus, and a non-transitory computer-readable storage medium storing a control program of an MRI apparatus.

### BACKGROUND

An MRI apparatus is an imaging apparatus that magnetically excites nuclear spin of an object placed in a static magnetic field by applying a radio frequency (RF) signal having the Larmor frequency and reconstructs a magnetic resonance (MR) image based on MR signals emitted from the object due to the excitation. An MRI apparatus can non-invasively acquire MR signals from the object.

In imaging with the use of an MRI apparatus, there is a technique that supplementarily uses an optical image generated by an optical camera.

In order to acquire MR signals from the object, a receiving coil such as a whole body (WB) coil and an RF coil is used to receive MR signals emitted from the object. There are various types of RF coils that receive MR signals near the object, such as those for the head, for the chest, for the spine, for the lower limbs, and for the whole body depending on an examination portion of the object (i.e., site or anatomical part of the object to be examined). In some cases, a plurality of RF coils can be used for the same examination portion of the object. For this reason, users such as a medical imaging technologist judge and select the RF coil(s) according to various information items such as the imaging purpose, the examination portion, and the medical condition of the patient, based on their own skills and experience. Since the users select the RF coil(s) based on their own skills and experience, the optimal RF coil is not necessarily selected in some cases.

### BRIEF DESCRIPTION OF THE DRAWINGS

FIG. 1 is a schematic diagram illustrating an overall configuration of an MRI apparatus according to one embodiment;

FIG. 2 is a flowchart illustrating operation of the MRI apparatus according to the embodiment;

FIG. 3 is a schematic diagram illustrating how an examination portion of an object is imaged by an optical camera in the MRI apparatus according to the embodiment;

FIG. 4 is a schematic diagram illustrating a flow of information until the MRI apparatus according to the embodiment selects at least one specific RF coil;

FIG. 5 is a schematic diagram illustrating a method for selecting the specific RF coil(s) in the MRI apparatus according to the embodiment;

FIG. 6 is a flowchart illustrating operation of selecting the specific RF coil(s) to be executed by the MRI apparatus according to the embodiment when the examination portion is the knee;

FIG. 7 is a schematic diagram illustrating a case of reflecting the posture of the object in the method for selecting the specific RF coil(s) in the MRI apparatus according to the embodiment;

FIG. 8 is a schematic diagram illustrating presentation of the selected specific RF coil(s) in the MRI apparatus according to the embodiment;

FIG. 9 is a schematic diagram illustrating an overall configuration of the MRI apparatus according to a modification of the embodiment; and

FIG. 10 is a flowchart illustrating operation of the MRI apparatus according to the modification of the embodiment.

### DETAILED DESCRIPTION

Hereinbelow, a detailed description will be given of respective embodiments of an MRI apparatus, a control method for an MRI apparatus, and a non-transitory computer-readable storage medium storing a control program of an MRI apparatus by using the accompanying drawings.

In one embodiment, an MRI apparatus comprising processing circuitry configured to: acquire an optical image of an object placed on a table of a bed; acquire information on an examination portion of the object; and select a specific RF coil to be used for imaging the examination portion of the object from a plurality of available RF coils based on the optical image(s) and information on the plurality of available RF coils specified from information on the examination portion.

### Overall Configuration of MRI Apparatus

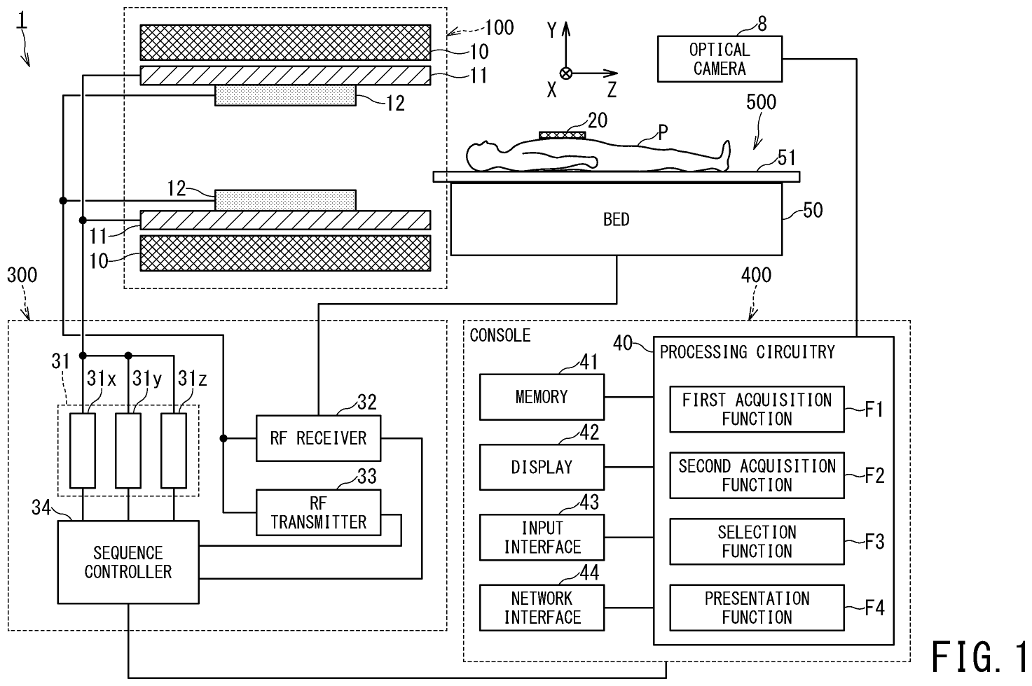

FIG. 1 illustrates an overall configuration of an MRI apparatus 1 according to one embodiment. The MRI apparatus 1 includes a gantry 100, a control cabinet 300, a console 400, a bed 500, and an optical camera 8.

The gantry 100 and the bed 500 are disposed in a shielded room called an examination room, for example. The control cabinet 300 is disposed in a machine room, for example. The console 400 is disposed in a control room, for example.

The gantry 100 includes, for example, a static magnetic field magnet 10, a gradient coil assembly 11, and a whole body (WB) coil 12, and these components are housed in a cylindrical housing. The bed 500 includes a bed body 50 and a table 51. The MRI apparatus 1 also includes at least one RF coil 20 disposed close to an object P. The RF coil 20 and the MRI apparatus 1 are configured to be connectable to each other. In detail, the RF coil 20 and the table 51 of the MRI apparatus 1 are configured to be connectable to each other.

The control cabinet 300 includes three gradient coil power supplies 31 (31x for the X-axis, 31y for the Y-axis, and 31z for the Z-axis), an RF receiver 32, an RF transmitter 33, and a sequence controller 34.

The static magnetic field magnet 10 of the gantry 100 is substantially in the form of a cylinder and generates a static magnetic field inside a bore, which is a space inside the cylindrical structure of the static magnetic field magnet 10 and is also an imaging region of the object P such as a patient. The static magnetic field magnet 10 includes a superconducting coil inside, and the superconducting coil is cooled down to an extremely low temperature by liquid helium. The static magnetic field magnet 10 generates a static magnetic field by applying an electric current provided from a static magnetic field power supply (not shown) to the superconducting coil in an excitation mode. Afterward, when the static magnetic field magnet 10 shifts to a persistent current mode, the static magnetic field power supply is disconnected. Once it shifts to the persistent current mode, the static magnetic field magnet 10 continues to generate a strong static magnetic field for a long time, for example, over one year. Note that the static magnetic field magnet 10 maybe configured as a permanent magnet.

The gradient coil assembly 11 is also substantially in the form of a cylinder and is fixed to the inside of the static magnetic field magnet 10. This gradient coil assembly 11 is composed of three gradient coils for the respective X-axis, Y-axis, and Z-axis. The three gradient coils generate gradient magnetic fields in the respective directions of the X-axis, Y-axis, and Z-axis by being supplied with gradient magnetic field currents from the respective gradient coil power supplies 31x, 31y, and 31z so as to apply the generated gradient magnetic fields to the object P.

The bed body 50 of the bed 500 can move the table 51 in the vertical direction and the horizontal direction. The bed body 50 moves the table 51 with the object P placed thereon to a predetermined height before imaging. When the object P is imaged, the bed body 50 moves the table 51 in the horizontal direction to move the object P to the inside of the bore.

The WB coil 12 is shaped substantially in the form of a cylinder so as to surround the object P and is fixed to the inside of the gradient coil assembly 11. The WB coil 12 applies RF pulses transmitted from the RF transmitter 33 to the object P, and receives MR signals emitted from the object P due to excitation of hydrogen nucleus.

The RF coil 20 receives MR signals emitted from the object P at a position close to the object P. There are various types of RF coils 20 depending on an examination portion of the object P, such as the head, the neck, the chest, the spine, the upper limbs, the lower limbs, and the whole body. FIG. 1 illustrates a state in which the RF coil 20 for the chest is attached to the object P.

The RF transmitter 33 transmits each RF pulse to the WB coil 12 based on an instruction from the sequence controller 34. The RF receiver 32 receives MR signals detected by the WB coil 12 and/or the RF coil 20, and transmits raw data obtained by digitizing the detected MR signals to the sequence controller 34.

The sequence controller 34 performs a scan of the object P by driving the gradient coil power supplies 31, the RF transmitter 33, and the RF receiver 32 under the control of the console 400. When the sequence controller 34 receives the raw data acquired by the scan from the RF receiver 32, the sequence controller 34 transmits the raw data to the console 400.

The sequence controller 34 includes processing circuitry (not shown). This processing circuitry is configured as a processor, which executes predetermined programs, or is configured as hardware such as a field programmable gate array (FPGA) and an application specific integrated circuit (ASIC), for example.

The console 400 is configured as a computer that includes processing circuitry 40, a memory 41, a display 42, and an input interface 43. The console 400 may include a network interface 44. The console 400 is one aspect of an image processing device. In addition, part or all of the image processing device may be achieved by a computer different from the console 400, such as a tablet computer.

The memory 41 is a recording medium including a read-only memory (ROM) and/or a random access memory (RAM) in addition to an external memory device such as a hard disk drive (HDD) and an optical disc device. The memory 41 stores various programs to be executed by the processor of the processing circuitry 40 as well as various data and information. The memory 41 stores a database in which a plurality of available RF coils 20 in the MRI apparatus are registered, such as information on a plurality of RF coils provided in a facility where the MRI apparatus is installed. Further, the memory 41 stores information on the priority order of the RF coils to be selected depending on the examination portion of the object P, for example.

The display 42 is a display device such as a liquid crystal display panel, a plasma display panel, and an organic EL panel. The input interface 43 includes various devices for a user to input various data and information, and is configured of a mouse, a keyboard, a trackball, and/or a touch panel, for example. The network interface 44 is a wired or wireless interface that allows communication with various devices connected to the network and exchange various data and information.

The processing circuitry 40 is a circuit provided with a central processing unit (CPU) and/or a special-purpose or general-purpose processor, for example. The processor implements various functions described below by executing the various programs stored in the memory 41. The processing circuitry 40 may be configured of hardware such as an FPGA and an ASIC. The various functions described below can also be implemented by such hardware. Additionally, the processing circuitry 40 can implement the various functions by combining hardware processing and software processing based on its processor and programs.

The console 400 controls the entirety of the MRI apparatus 1 by using each of these components. The processing circuitry 40 causes the sequence controller 34 to perform a scan based on the inputted imaging conditions, and then reconstructs an MR image based on the raw data inputted from the sequence controller 34, i.e., digitized MR signals. The reconstructed MR image is displayed on the display 42 or stored in the memory 41.

The optical camera 8 is installed on the ceiling of the imaging room where the MRI apparatus 1 is installed, for example. The optical camera 8 includes an optical lens, an image sensor, an amplifier, an A/D (Analog to Digital) converter (not shown), for example. The optical lens is an optical element that refracts and focuses light. The image sensor images an imaging target through an objective optical system. The amplifier amplifies a video signal outputted from the image sensor. The A/D converter converts an analog video signal outputted from the amplifier into a digital signal. The optical camera 8 is connected to the processing circuitry 40, and the generated optical image is outputted as a digital signal to the processing circuitry 40.

The optical camera 8 images all or part of the table 51 before entering the gantry 100 from above, and generates an optical image including (i.e., depicting) the examination portion of the object P placed on the table 51. For example, the optical camera 8 can generate optical images including at least the examination portion of the object P as a moving image by time-sequentially imaging the object P at a predetermined frame rate.

Note that the optical lens of the optical camera 8 may 8 maybe a standard lens or may be a so-called wide-angle lens that has a wider angle of view than the standard lens. Further, the optical camera 8 is not limited to the aspect of being installed on the ceiling, but may be fixed to a cover that covers the gantry 100, or may be attached to the gantry 100 or the wall around the gantry 100.

### Operation of MRI Apparatus

As shown in FIG. 1, the processing circuitry 40 of the MRI apparatus 1 according to the embodiment implements a first acquisition function F1, a second acquisition function F2, a selection function F3, and a presentation function F4. Note that at least one or all of the above-described functions may be provided in a computer different from the console 400, such as a tablet computer.

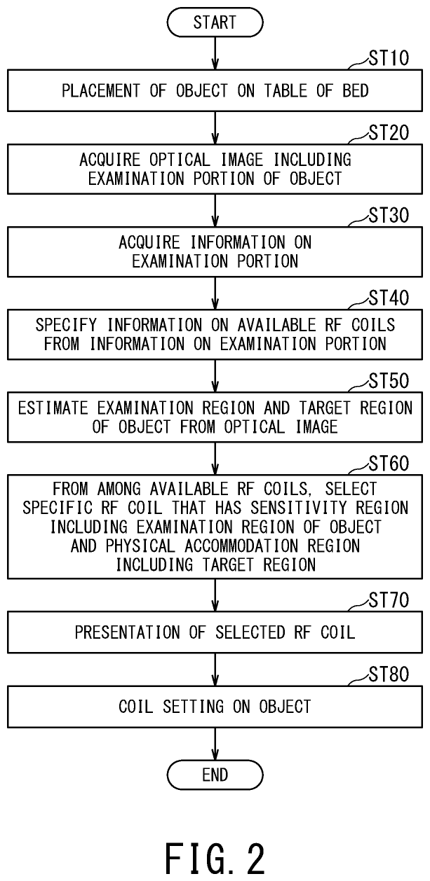

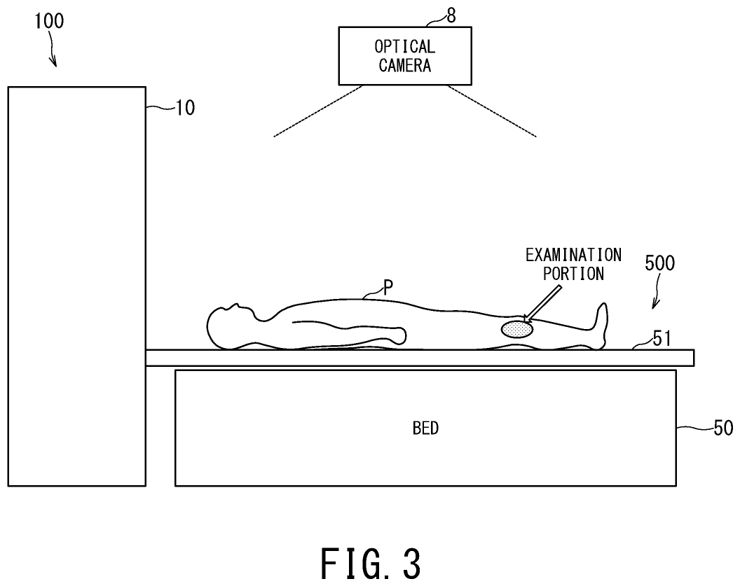

Next, a description will be given of the configuration and operation of the respective functions of the processing circuitry 40 based on a flowchart of FIG. 2 illustrating the operation of the MRI apparatus 1 according to the embodiment or its control program by referring to FIG. 3 to FIG. 8 as required.

In the step ST10, the object P is placed on the table 51 of the bed 500.

In the step ST20, the first acquisition function F1 acquires an optical image generated by imaging the object P placed on the table 51. As shown in FIG. 3, the optical image including the examination portion of the object P placed on the table 51 is generated by the optical camera 8.

Returning to FIG. 2, in the step ST30, the second acquisition function F2 acquires information on the examination portion of the object P. The information on the examination portion of the object P can be acquired by: (a) a user's input operation via the input interface 43 and/or (b) reading out information stored in advance in the memory 41 based on the doctor's order, the examination type, and/or the examination name, for example. Note that the information on the examination portion of the object P may be acquired before the step ST10.

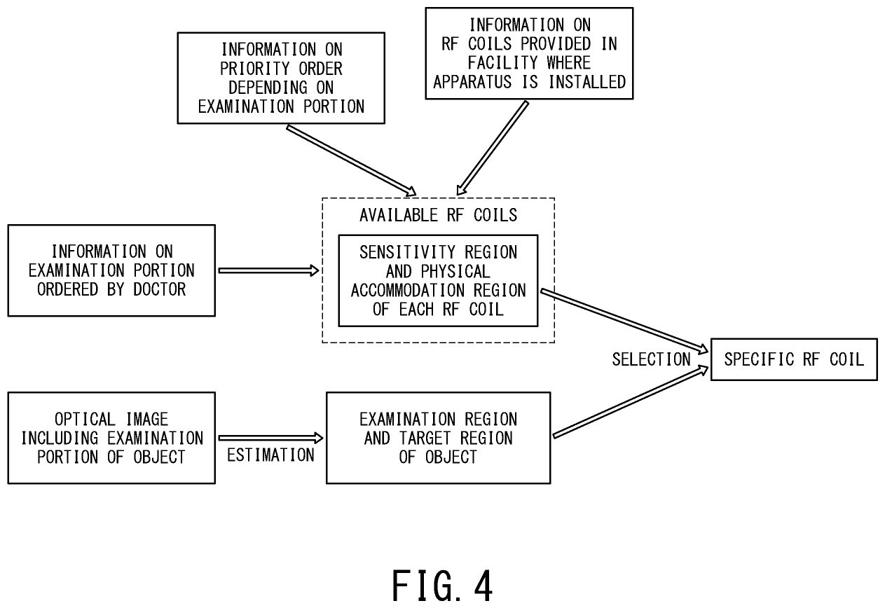

In the step ST40, the selection function F3 specifies the information on the plurality of available RF coils 20 from the information on the examination portion. FIG. 4 illustrates a flow of information until the MRI apparatus 1 according to the embodiment selects at least one or necessary number of specific RF coil(s). As shown in FIG. 4, in addition to the information on the examination portion itself, the information on the plurality of available RF coils 20 specified from the information on the examination portion may further include: (a) information on the plurality of RF coils 20 provided in the facility where the MRI apparatus 1 is installed, (b) information on the plurality of RF coils 20 that are compatible with the model of the MRI apparatus 1, and (c) information on the priority order of the specific RF coil(s) to be selected, for example.

For example, the plurality of available RF coils 20 are specified on the basis of the information on the plurality of RF coils 20 provided in the facility where the MRI apparatus 1 is installed, and the information on the priority order of specific RF coils to be selected. The information on the plurality of available RF coils 20 is stored in the memory 41, for example.

For example, even if the provided or prepared RF coils 20 differ depending on the installation facility such as a hospital or even if the available RF coils 20 differ depending on the model of the MRI apparatus 1, the specific RF coil(s) can be selected in the step ST60 described below from among the plurality of stored available RF coils 20 by using the information on the plurality of RF coils 20 provided in the facility where the MRI apparatus 1 is installed. In this manner, the selection function F3 can select the specific RF coil(s) from among the plurality of RF coils 20 provided in the facility where the MRI apparatus 1 is installed.

For example, when the information on the specific RF coil(s) for obtaining a high-quality MR image in accordance with the examination portion of the object P is used as the information on the priority order of the specific RF coil(s), in the step ST60 for selecting the specific RF coil(s), the selection function F3 can select the specific RF coil(s) in the order in which an RF coil capable of obtaining a higher-quality MR image is selected with higher priority. In this manner, the selection function F3 can select the specific RF coil(s) from the plurality of available RF coils in descending order of priority, by applying the information on the priority order of the specific RF coil(s), for example.

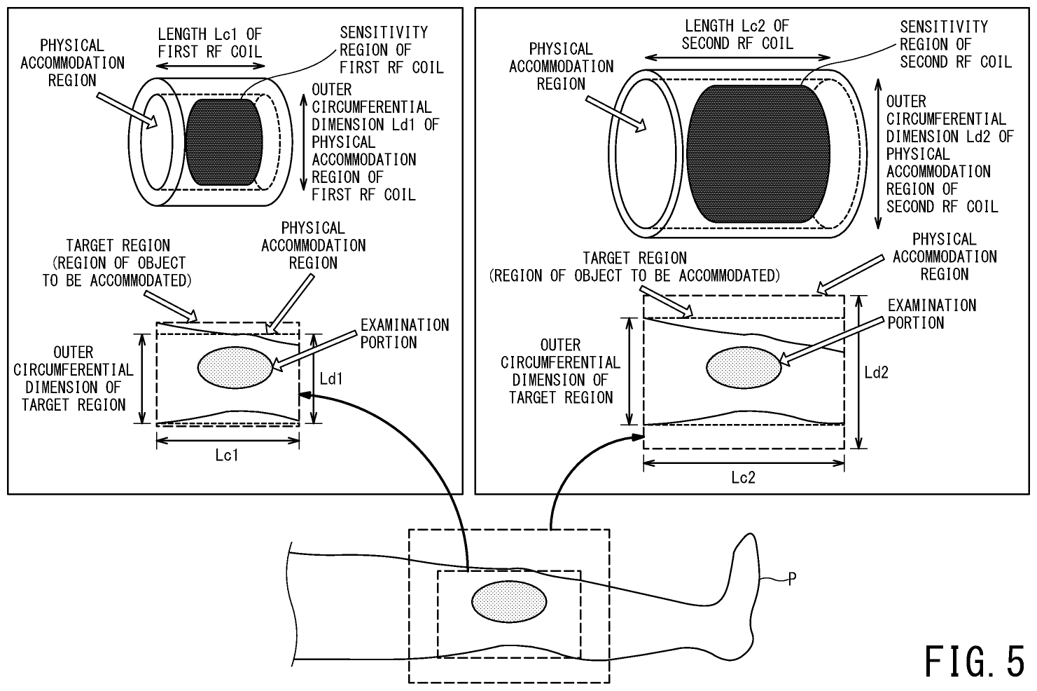

Each of the plurality of RF coils 20 includes information on a sensitivity region and a physical accommodation region, as the information on the RF coil 20. FIG. 5 illustrates the sensitivity region and physical accommodation region of the RF coil 20, and also illustrates an examination region and a target region of the object P. As shown in FIG. 5, the sensitivity region of the RF coil 20 is a region where MR signals from the object P can be detected with satisfactory sensitivity in terms of image reconstruction. The physical accommodation region of the RF coil 20 is the region where the RF coil 20 can physically accommodate the examination portion of the object P. For example, when the RF coil 20 has a cylindrical shape as shown in FIG. 5, the physical accommodation region is a substantially columnar region defined by its inner circumference and length.

The examination region of the object is a region that includes the examination portion of the object, and can also be referred to as the imaging region of interest. The target region of the object is a region of the object to be accommodated in the RF coil 20. Even if the target region of the object is the same as the examination portion or examination region of the object, the size and shape of the target region of the object vary depending on the body shape of the object, the posture of the object, or the bending degree of a joint in the case where the examination portion includes the joint, for example.

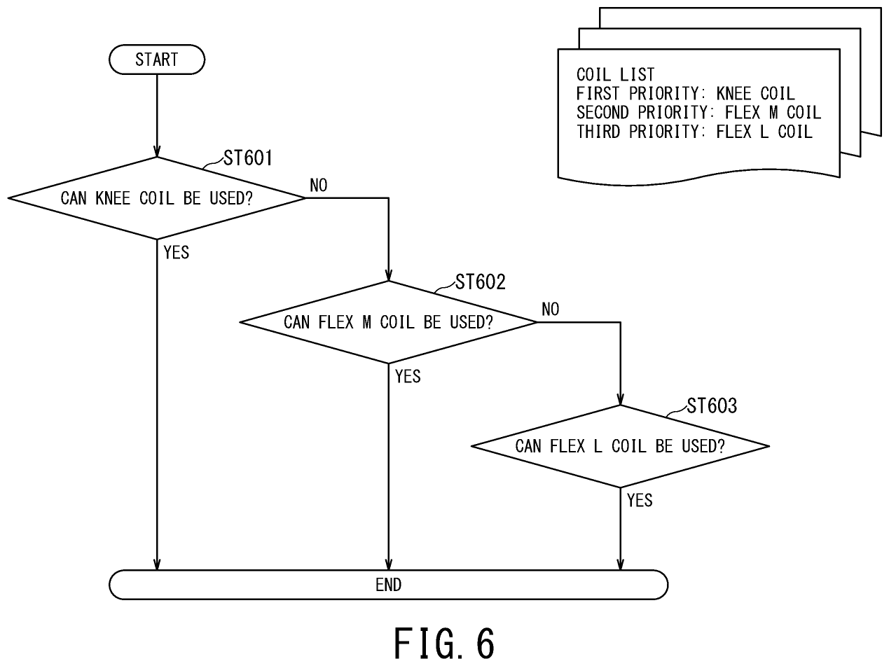

The information on the plurality of available RF coils 20 is based on both the information on the priority order of the specific RF coil(s) to be selected and the information on the plurality of RF coils 20 provided in the facility where the MRI apparatus 1 is installed, for example. The information on the plurality of available RF coils 20 maybe stored in the form of a coil list configured as a data table that includes information on the sensitivity region and the physical accommodation region of each of the plurality of available RF coils 20 (FIG. 6), for example.

Returning to FIG. 2, in the step ST50, the selection function F3 uses the optical image to estimate the examination region including the specific examination portion of the object P and the target region of the object P to be accommodated in the specific RF coil. As for the estimation timing, the estimation may be started after the movement of the object P has stopped as detected by a known movement detection technique with the use of information amount and amount of change of the image, for example.

In the step ST60, the selection function F3 selects at least one or necessary number of specific RF coil(s) to be used for the examination portion of the object P from among the plurality of available RF coils 20 based on the optical image and the information on the plurality of available RF coils 20.

The selection function F3 selects the specific RF coil(s) based on the estimated examination region and the target region. As the specific RF coil(s), the selection function F3 selects the RF coil 20 that satisfies predetermined two conditions as follows. Firstly, the sensitivity region of the RF coil 20 includes the examination region of the object P. Secondly, the physical accommodation region of the RF coil 20 includes the target region of the object P.

By referring to FIG. 5 illustrating a selection method for the specific RF coil(s), a description will be given of the examination region and the target region of the object P, and the sensitivity region and the physical accommodation region of both of the first RF coil and the second RF coil, in detail. Even if the sensitivity region of an RF coil 20 includes the examination region of the object P as in the case of the first RF coil shown in the left part of FIG. 5, the physical accommodation region of this RF coil 20 may not include the target region depending on the physique of the object P and the thickness and shape of the examination portion, for example.

For example, whether or not the physical accommodation region of the RF coil 20 includes the target region of the object P may be determined depending on: (a) whether the outer circumferential dimension of the target region of the object P can be included in the outer circumferential dimension of the physical accommodation region of the first RF coil or not, (b) whether the outer diameter dimension of the target region of the object P can be included in the outer diameter dimension of the physical accommodation region of the first RF coil or not, or (c) another known method. In the MRI apparatus 1 according to the embodiment, when the physical accommodation region of an RF coil does not include the target region of the object P like the first RF coil shown in the left part of FIG. 5, this RF coil is not selected as the specific RF coil.

In the MRI apparatus 1 according to the embodiment, when the sensitivity region of an RF coil does not include the examination region of object P, this RF coil is not selected either as the specific RF coil.

In other words, in either case where the physical accommodation region of an RF coil does not include the target region of object P or where the sensitivity region of an RF coil does not include the examination region of object P, this RF coil is not selected as the specific RF coil.

As the predetermined two conditions, when (i) the physical accommodation region of an RF coil includes the target region of the object P and (ii) the sensitivity region of this RF coil includes the examination region of object P, this RF coil can be selected as the specific RF coil. For example, as shown in the right part of FIG. 5, the sensitivity region of the second RF coil includes the examination region of the object P, and the physical accommodation region of the second RF coil includes the target region of the object P. In this case, the MRI apparatus 1 according to the embodiment selects the second RF coil as the specific RF coil. However, when this second RF coil does not meet the specifications of the MRI apparatus 1 or when the facility where the MRI apparatus 1 is installed does not have the second RF coil, the second RF coil is not selected as the specific RF coil.

FIG. 6 is a flowchart illustrating the operation of selecting the specific RF coil(s) to be executed by the MRI apparatus 1 when the examination portion is the knee. For example, when the information on the priority order of the specific RF coil(s) to be selected is stored, such as the case where the first priority is the RF coil for the knee, the second priority is the RF coil of Flex-M size, and the third priority is the RF coil of Flex-L size as shown in FIG. 6, whether the first RF coil and the second RF coil should be selected can be sequentially determined in the order of priority, i.e., in the order of the step ST601, the step ST602, and the step ST603. In FIG. 6, if the determination result of the step ST603 is NO, i.e., if none of the RF coil can be selected as the specific RF coil, the information indicating absence or non-existence of the selectable specific RF coil can be presented to a user.

If the determination regarding the above-described two conditions is performed sequentially according to the priority order, it is not necessarily required to perform determination on all the available RF coils 20 that are the choices for the specific RF coil(s), thus, the number of RF coils to be subjected to the determination can be almost minimized, and consequently, the specific RF coil(s) can be selected efficiently.

Among the plurality of RF coils 20 provided in the facility where the MRI apparatus 1 is installed, the RF coil with higher priority order depending on the examination portion is first subjected to the determination as to whether this RF coil satisfies both conditions, and the selection function F3 can select the specific RF coil(s) satisfying both conditions by sequentially performing this determination in the order of priority. In this manner, the selection function F3 can select the specific RF coil(s) from the plurality of the plurality of RF coils 20 provided in the facility in the order of priority.

Further, the selection function F3 may estimate, from the optical image, at least one of the examination region and the target region where the posture of the object P placed on the table 51 and the bending degree of a joint if it is included in the examination portion are reflected in the estimation.

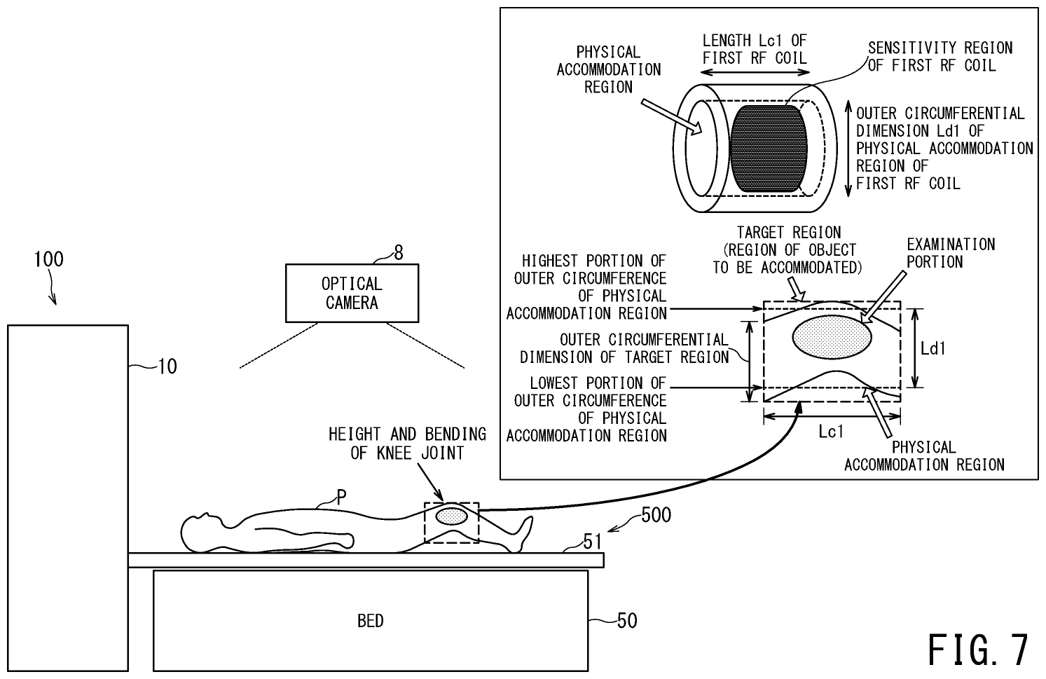

In the method for selecting the specific RF coil(s), reflecting the condition such as the posture of the object P will be described by using FIG. 7 where the knee joint of the object P is bent.

As shown in FIG. 7, for example, even when the outer circumference dimension of the target region of the object P is smaller than the inner circumference dimension of the physical accommodation region of the RF coil 20 in the case where the object P cannot bend the knee joint, the target region of the object P may not be included in the physical accommodation region of the RF coil 20, as exemplified by (i) a case where the height of the knee joint is higher than the highest portion of the outer circumference of the physical accommodation region of the RF coil 20 and (ii) another case where the height of the thigh or sura is lower than the lowest portion of the outer circumference of the physical accommodation region of the RF coil 20.

Furthermore, in some cases where the object P is in the same state, the examination region of the object P is outside the range of the sensitivity region of the RF coil 20 i.e., not included in the sensitivity region of the RF coil 20. In such a case, when the examination region and the target region that reflect the posture of the object P and the bending degree of the joint if it is included in the examination portion can be estimated, the optimal specific RF coil can be selected without imposing a burden on the object P.

In a follow-up examination, instead of performing the steps ST40, ST50, and ST60, the processing circuitry 40 can read out the past MR image data and select the same RF coil that was used at the time of acquiring the past MR image as the specific RF coil.

Returning to FIG. 2, in the step ST70, the presentation function F4 presents the selected RF coil to the user. Further, the presentation function F4 may present the selected RF coil to the user in such a manner that the user can understand or grasp the coil setting position of the selected RF coil.

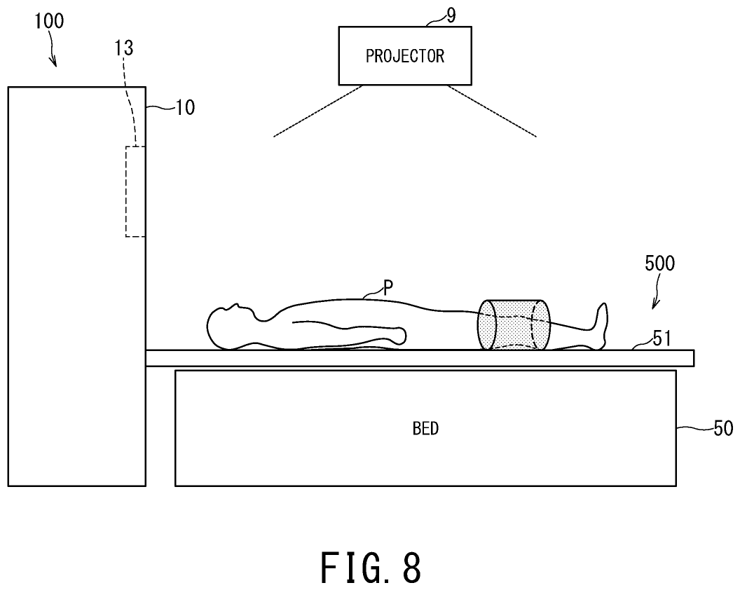

FIG. 8 is a schematic diagram illustrating a case of presentation of the selected specific RF coil(s) in the MRI apparatus 1 according to the embodiment. As shown in FIG. 8, the selected specific RF coil(s) may be presented to the user as visual information, such as display on a monitor 13 and projection display by a projector 9.

The monitor 13 is installed in the examination room of the MRI apparatus 1 at a location where the monitor 13 can be viewed by the user. The monitor 13 maybe fixed to the cover that covers the gantry 100 or may be attached to the gantry 100 or the wall around the gantry 100, for example. The monitor 13 is configured of a general display output device such as a liquid crystal display and an OLED (Organic Light Emitting Diode) display. For example, the selected specific RF coil(s) may be presented in such a manner that each selected specific RF coil is superimposed on the optical image of the object P and displayed on the monitor 13 such that the coil setting position is distinguishable or understandable. Furthermore, the monitor 13 maybe provided with the input interface 43 such as a touch panel.

The projector 9 is configured of a display output device capable of projection, such as a liquid crystal projector, a video display system using a digital micromirror device, a reflective liquid crystal element projector, and a laser projector. For example, the selected specific RF coil(s) may be presented as projection display on the object P by the projector 9 such that the coil setting position is distinguishable or understandable.

In the step ST80, coil setting is performed on the object P. The user can perform the coil setting based on the selected specific RF coil(s) and the coil setting position. After the coil setting, a diagnostic scan and/or a pre-scan is/are performed.

According to the MRI apparatus 1 of the embodiment, the specific RF coil(s) suitable for the examination portion of the object can be selected based on the optical image generated by the optical camera. Thus, even when a user with poor skill and few experience performs the coil setting, the optimal RF coil can be selected from among the plurality of available RF coils 20. Further, high-quality MR images can be obtained by selecting the optimal RF coil. Since there is no need to repeatedly perform the coil setting on the object P, the burden on the object P is reduced.

### Modification

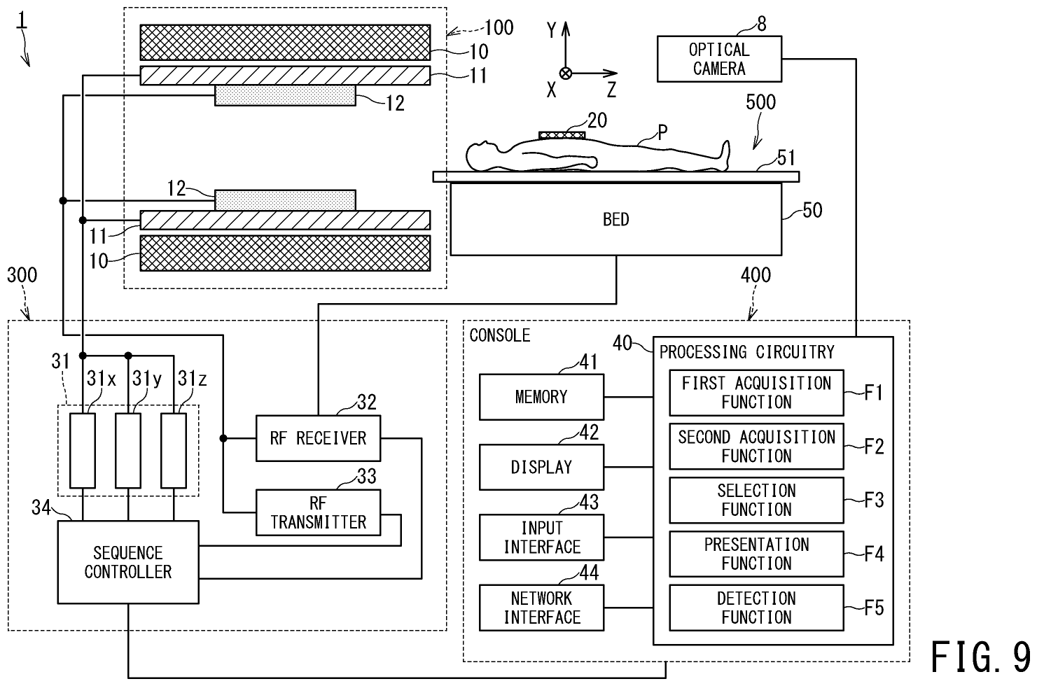

FIG. 9 is a schematic diagram illustrating an overall configuration of the MRI apparatus 1 according to a modification of the embodiment. As shown in FIG. 9, the MRI apparatus 1 according to the modification of the embodiment differs from the above-described embodiment in that the processing circuitry 40 further includes a detection function F5 of detecting the movement of the object P from the optical images. Since the other configurations are not substantially different from the embodiment shown in FIG. 1, the same reference signs are given to the same components and duplicate description is omitted.

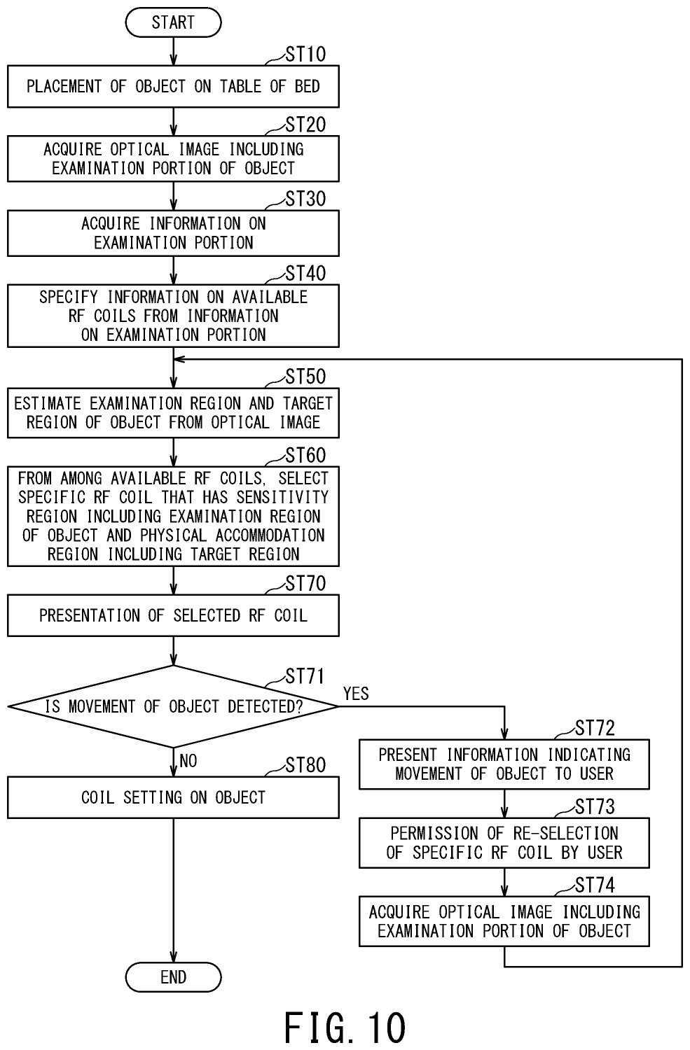

FIG. 10 is a flowchart illustrating the operation of the MRI apparatus 1 according to the modification of the embodiment. The modification of the embodiment differs from the embodiment in that the movement of the object P is detected and the information that the object P has moved is presented to the user. The steps ST10, ST20, ST30, ST40, ST50, ST60, and ST70 are not substantially different from the embodiment shown in FIG. 2 and are assigned with the same reference signs, and duplicate description is omitted. Although the step ST71 may be performed immediately after the step ST50 or ST60, a description will be given of the case where the processing proceeds to the step ST71 after the step ST70.

In the step ST71, the detection function F5 detects whether the object P has moved or not. If there is no movement of the object P (NO in the step ST71 of FIG. 10), the processing proceeds to the step ST80. Since the step ST80 is not substantially different from the embodiment shown in FIG. 2, the same reference sign is assigned, and duplicate description is omitted.

If there is movement of the object P (YES in the step ST71 of FIG. 10), the processing proceeds to the step ST72.

In the step ST72, the presentation function F4 presents, to the user, the information that object P has moved. In other words, if the detection function F5 detects the movement of the object P after selection of the specific RF coil(s) and before setting of the specific RF coil, the information that object P has moved is present to the user. Further, at the timing that is posterior to estimation of the examination region and the target region of the object from the optical image and is prior to setting of the specific RF coil(s), the detection function F5 may detect the movement of the object P and cause the presentation function F4 to present this information to the user.

The information may be presented to the user as visual information, such as display on the monitor 13, or as audio information. In addition, in the presentation of the information that the object P has moved, the presentation function F4 may present: (a) information that the specific RF coil(s) is/are automatically re-selected; and/or (b) information that the user can choose to permit or prohibit re-selection of the specific RF coil(s).

If the user chooses permission of re-selection of the specific RF coil(s) in the step ST73, in the next step ST74, the first acquisition function F1 acquires the optical image of the object P placed on the table 51. The permission by the user is inputted via the monitor 13 or a tablet computer, for example. Since the step ST74 is not substantially different from the step ST20, duplicate description is omitted. After the step ST74, the processing proceeds to the step ST50.

According to the MRI apparatus 1 of the modification of the embodiment, if the object P moves after estimation of the examination region and the target region of the object from the optical image and before setting of the specific RF coil(s), the appropriate specific RF coil(s) can be re-selected from among the plurality of available RF coils 20.

According to the MRI apparatus, the method for controlling the MRI apparatus, and the non-transitory storage medium for the control program of the MRI apparatus of at least one embodiment described above, the specific RF coil(s) suitable for the examination portion of the object P can be selected on the basis of the optical image generated by the optical camera.

In the above-described embodiments, the term “processor” means a circuit such as a special-purpose or general purpose CPU, a GPU (Graphics Processing Unit), an ASIC, a programmable logic device including an SPLD (Simple Programmable Logic Device) and a CPLD (Complex Programmable Logic Device), and an FPGA, for example. When the processor is a CPU, for example, the processor implements various functions by reading in and executing the programs (i.e., the control programs of the MRI apparatus) stored in the memory.

In addition, when the processor is an ASIC, for example, instead of storing the programs in the memory, the functions corresponding to the programs are directly incorporated in the circuit of the processor as a logic circuit. In this case, the processor implements various functions through hardware processing in which the processor reads in and executes the programs incorporated into the circuit. Additionally or alternatively, the processor can also achieve various functions by combining software processing and hardware processing.

Although a description has been given of the case where a single processor of the processing circuitry 40 achieves the respective functions in the above-described embodiments, the processing circuitry 40 may be configured by combining a plurality of independent processors in such a manner that each processor implements each function. Further, when a plurality of processors are provided, a memory for storing the programs may be provided for each processor or a single memory may collectively store the programs corresponding to the functions of all the processors.

While certain embodiments have been described, these embodiments have been presented by way of example only, and are not intended to limit the scope of the invention. Indeed, the novel methods and systems described herein may be embodied in a variety of other forms; furthermore, various omissions, substitutions and changes in the form of the methods and systems described herein may be made without departing from the scope of the invention as defined by the appended claims.

What is claimed is:

1. An MRI apparatus comprising processing circuitry configured to:
acquire an optical image of an object placed on a table of a bed;
acquire information on an examination portion of the object; and
select a specific RF coil to be used for imaging the examination portion of the object from a plurality of available RF coils based on the optical image and information on the plurality of available RF coils specified from the information on the examination portion.

2. The MRI apparatus according to claim 1, further comprising a memory configured to store information on a plurality of RF coils provided in a facility as the plurality of available RF coils, the facility being a place where the MRI apparatus is installed,
wherein the processing circuitry is configured to select the specific RF coil from the plurality of RF coils provided in the facility.

3. The MRI apparatus according to claim 1, further comprising a memory configured to store information on priority order for selecting the specific RF coil depending on the examination portion of the object,
wherein the processing circuitry is configured to select the specific RF coil from the plurality of available RF coils based on the priority order.

4. The MRI apparatus according to claim 2,
wherein the memory is further configured to store information on priority order for selecting the specific RF coil depending on the examination portion of the object,
wherein the processing circuitry is further configured to select the specific RF coil from the plurality of available RF coils based on the priority order.

5. The MRI apparatus according to claim 1, wherein the information on the plurality of available RF coils includes information on:
a sensitivity region where magnetic resonance signals from the object can be detected with satisfactory sensitivity in terms of image reconstruction; and
a physical accommodation region that is part of an RF coil and can physically accommodate the examination portion of the object.

6. The MRI apparatus according to claim 1, wherein the processing circuitry is configured to:
estimate an examination region and a target region of the object from the optical image, wherein the examination region includes the specific examination portion of the object, and the target region is a region to be accommodated in the specific RF coil; and
select the specific RF coil based on the examination region and the target region.

7. The MRI apparatus according to claim 6, wherein the processing circuitry is configured to select the specific RF coil that includes:
a sensitivity region including the examination region of the object; and
a physical accommodation region including the target region of the object.

8. The MRI apparatus according to claim 6, wherein, in estimation of at least one of the examination region and the target region from the optical image, the processing circuitry reflects posture of the object placed on the table and bending degree of a joint if it is included in the examination portion.

9. The MRI apparatus according to claim 1, wherein the processing circuitry is configured to present at least one of a selected specific RF coil and a coil setting position of the selected specific RF coil to a user by using at least one of monitor display and projection display.

10. The MRI apparatus according to claim 6, wherein the processing circuitry is configured to:
detect movement of the object from the optical image; and
present detected information indicating that the object has moved to a user in a case of detecting the movement of the object after estimation of the examination region and the target region of the object from the optical image and before setting of the specific RF coil.

11. A non-transitory computer-readable storage medium storing a control program for causing an MRI apparatus to execute processing comprising:
an acquisition process of acquiring an optical image of an object placed on a table of a bed and information on an examination portion of the object; and
a selection process of selecting a specific RF coil to be used for imaging the examination portion of the object from a plurality of available RF coils based on the optical image and information on the plurality of available RF coils specified from the information on the examination portion.

12. A control method for an MRI apparatus comprising steps of:
acquiring an optical image of an object placed on a table of a bed;
acquiring information on an examination portion of the object; and
selecting a specific RF coil to be used for imaging the examination portion of the object from a plurality of available RF coils based on the optical image and information on the plurality of available RF coils specified from the information on the examination portion.
